(19) United States
(12) Patent Application Publication
(10) Pub. No.: US 2025/0152035 A1
(43) Pub. Date: May 15, 2025

(54) FLEX CIRCUIT WITH LONGITUDINALLY-SPACED POSITION SENSORS FOR STEERABLE MEDICAL INSTRUMENT

(71) Applicants: Acclarent, Inc., Irvine, CA (US); Biosense Webster (Israel) LTD., Yokneam (IL)

(72) Inventors: Carly N. Harad, Irvine, CA (US); Shmuel Auerbach, Kerem Maharal (IL)

(21) Appl. No.: 18/937,813

(22) Filed: Nov. 5, 2024

Related U.S. Application Data
(60) Provisional application No. 63/547,995, filed on Nov. 10, 2023.

(51) Int. Cl. A61B 5/06; A61B 1/05; A61B 1/06; A61M 25/01

(52) U.S. Cl. CPC A61B 5/062; A61B 1/05; A61B 1/0676; A61M 25/0127

(57) ABSTRACT

An apparatus includes a shaft assembly having a proximal portion and a distal portion. The distal portion is configured to enable lateral deflection of the distal end relative to a longitudinal axis defined by the proximal portion. A distal navigation sensor positioned at or near the distal end of the distal portion is configured to indicate a position of the distal end of the flexible distal portion in three-dimensional space. A pair of proximal navigation sensors positioned at or near a proximal end of the flexible distal portion are configured to indicate a roll orientation of the proximal end of the flexible distal portion about the longitudinal axis.

This application claims priority under 35 U.S.C. § 119 to U.S. Patent Application Ser. No. 63/547,995, which was filed on Nov. 10, 2023 and is incorporated herein by reference.

### BACKGROUND

Image-guided surgery (IGS) is a technique where a computer is used to obtain a real-time correlation of the location of an instrument that has been inserted into a patient's body to a set of preoperatively obtained images (e.g., a CT or MRI scan, 3-D map, etc.), such that the computer system may superimpose the current location of the instrument on the preoperatively obtained images. An example of an electromagnetic IGS navigation system that may be used in IGS procedures is the CARTO® 3 System by Biosense-Webster, Inc., of Irvine, California. In some IGS procedures, a digital tomographic scan (e.g., CT or MRI, 3-D map, etc.) of the operative field is obtained prior to surgery. A specially programmed computer is then used to convert the digital tomographic scan data into a digital map. During surgery, special instruments having sensors (e.g., electromagnetic coils that emit electromagnetic fields and/or are responsive to externally generated electromagnetic fields) are used to perform the procedure while the sensors send data to the computer indicating the current position of each surgical instrument. The computer correlates the data it receives from the sensors with the digital map that was created from the preoperative tomographic scan. The tomographic scan images are displayed on a video monitor along with an indicator (e.g., crosshairs or an illuminated dot, etc.) showing the real-time position of each surgical instrument relative to the anatomical structures shown in the scan images. The surgeon is thus able to know the precise position of each sensor-equipped instrument by viewing the video monitor even if the surgeon is unable to directly visualize the instrument itself at its current location within the body.

In some instances, it may be desirable to dilate an anatomical passageway in a patient. This may include dilation of ostia of paranasal sinuses (e.g., to treat sinusitis), dilation of the larynx, dilation of the Eustachian tube, dilation of other passageways within the ear, nose, or throat, etc. One method of dilating anatomical passageways includes using a guide wire and catheter to position an inflatable balloon within the anatomical passageway, then inflating the balloon with a fluid (e.g., saline) to dilate the anatomical passageway. For instance, the expandable balloon may be positioned within an ostium at a paranasal sinus and then be inflated, to thereby dilate the ostium by remodeling the bone adjacent to the ostium, without requiring incision of the mucosa or removal of any bone. The dilated ostium may then allow for improved drainage from and ventilation of the affected paranasal sinus.

It may also be desirable to ablate tissue within the ear, nose, or throat of a patient. For instance, such ablation may be desirable to remodel tissue (e.g., to reduce the size of a turbinate), to provide denervation (e.g., to disable the posterior nasal nerve), and/or for other purposes. Some such ablation treatments may include radiofrequency (RF) ablation with alternating current (AC) electrical energy; and/or irreversible electroporation (IRE) via pulsed field direct current (DC) electrical energy. To achieve ablation, an end effector with one or more needle electrodes or other kind(s) of tissue contacting electrodes may be activated with monopolar or bipolar electrical energy. Such ablation procedures may be carried out in conjunction with a dilation procedure or separately from a dilation procedure.

It may also be desirable to provide easily controlled placement of a dilation catheter, ablation instrument, or other ENT instrument in an anatomical passageway, including in procedures that will be performed only by a single operator. While several systems and methods have been made and used to position an ENT instrument in an anatomical passageway, it is believed that no one prior to the inventors has made or used the invention described in the appended claims.

### BRIEF DESCRIPTION OF THE DRAWINGS

The drawings and detailed description that follow are intended to be merely illustrative and are not intended to limit the scope of the invention as contemplated by the inventors.

FIG. 1 is a schematic view of an example of a surgery navigation system being used on a patient seated in an example of a medical procedure chair;

FIG. 2A is a front perspective view of an example of an instrument with a slider in a proximal position, such that a working element shaft of the instrument is retracted proximally relative to an open distal end of a shaft assembly of the instrument;

FIG. 2B is a front perspective view of the instrument of FIG. 2A, with the slider in a distal position, such that the working element shaft is extended distally relative to the open distal end of the shaft assembly;

FIG. 3 is a fragmentary front perspective view of a distal portion of the instrument of FIG. 2A, with an example of a distal endoscope cap removably attached to the open distal end of the shaft assembly;

FIG. 4A is a fragmentary front perspective view of the distal portion of the instrument of FIG. 2A, with an example of a flexible navigation sensor assembly attached to a flexible distal portion of the shaft assembly, showing the flexible distal shaft portion of the instrument in a straight configuration, and further showing the flexible navigation sensor assembly disposed along an outer cylindrical surface of the flexible distal shaft portion in a first curved configuration;

FIG. 4B is a fragmentary front perspective view of the distal portion of the instrument of FIG. 4A, showing the flexible distal shaft portion of the instrument in a bent configuration, and further showing the flexible navigation sensor assembly disposed along the outer cylindrical surface of the flexible distal shaft portion in a second curved configuration; and

FIG. 5 is a top plan view of the flexible navigation sensor assembly of FIG. 4A in a flat configuration.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

For clarity of disclosure, the terms “proximal” and “distal” are defined herein relative to a surgeon, or other operator, grasping a surgical instrument having a distal surgical end effector. The term “proximal” refers to the position of an element arranged closer to the surgeon, and the term “distal” refers to the position of an element arranged closer to the surgical end effector of the surgical instrument and further away from the surgeon. Moreover, to the extent that spatial terms such as “upper,” “lower,” “vertical,” “horizontal,” or the like are used herein with reference to the drawings, it will be appreciated that such terms are used for exemplary description purposes only and are not intended to be limiting or absolute. In that regard, it will be understood that surgical instruments such as those disclosed herein may be used in a variety of orientations and positions not limited to those shown and described herein.

As used herein, the terms “about” and “approximately” for any numerical values or ranges indicate a suitable dimensional tolerance that allows the part or collection of components to function for its intended purpose as described herein.

### I. Example of an Image Guided Surgery Navigation System

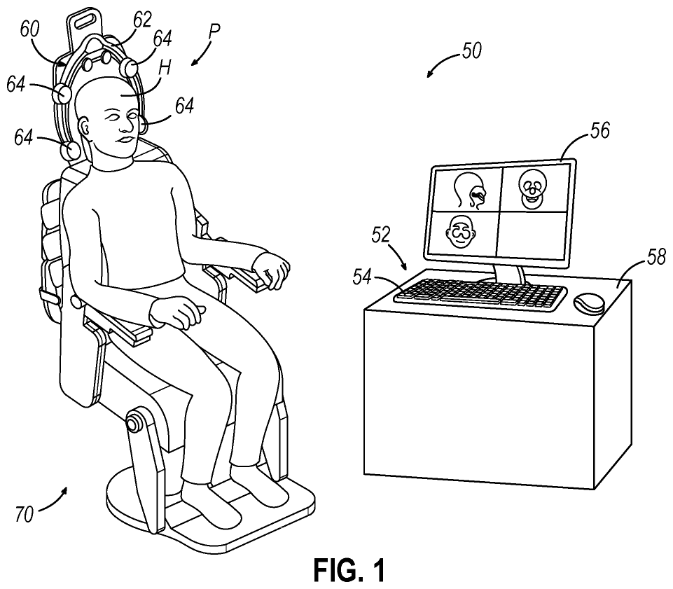

When performing a medical procedure within a head of a patient (P), it may be desirable to have information regarding the position of an instrument within the head (H) of the patient (P), particularly when the instrument is in a location where it is difficult or impossible to obtain an endoscopic view of a working element of the instrument within the head of the patient (P). FIG. 1 shows an example of an IGS navigation system 50 enabling a medical procedure to be performed within a head (H) of a patient (P) using image guidance. In addition to or in lieu of having the components and operability described herein the IGS navigation system 50 may be constructed and operable in accordance with at least some of the teachings of U.S. Pat. No. 7,720,521, entitled “Methods and Devices for Performing Procedures within the Ear, Nose, Throat and Paranasal Sinuses,” issued May 18, 2010, the disclosure of which is incorporated by reference herein, in its entirety; and/or U.S. Pat. No. 11,065,061, entitled “Systems and Methods for Performing Image Guided Procedures within the Ear, Nose, Throat and Paranasal Sinuses,” issued Jul. 20, 2021, the disclosure of which is incorporated by reference herein, in its entirety.

The IGS navigation system 50 of the present example includes a field generator assembly 60, which includes a set of magnetic field generators 64 that are integrated into a horseshoe-shaped frame 62. The field generators 64 are operable to generate alternating magnetic fields of different frequencies around the head (H) of the patient (P). An instrument may be inserted into the head (H) of the patient (P). Such an instrument may include one or more position sensors as described in greater detail below. In the present example, the frame 62 is mounted to a chair 70, with the patient (P) being seated in the chair 70 such that the frame 62 is located adjacent to the head (H) of the patient (P). By way of example only, the chair 70 and/or the field generator assembly 60 may be configured and operable in accordance with at least some of the teachings of U.S. Pat. No. 10,561,370, entitled “Apparatus to Secure Field Generating Device to Chair,” Issued Feb. 18, 2020, the disclosure of which is incorporated by reference herein, in its entirety. In some other variations, the patient (P) lies on a table; and the field generator assembly 60 is positioned on or near the table.

The IGS navigation system 50 of the present example further includes a processor 52, which controls the field generators 64 and other elements of the IGS navigation system 50. For instance, the processor 52 is operable to drive the field generators 64 to generate alternating electromagnetic fields; and process signals from the instrument to determine the location of a navigation sensor in the instrument within the head (H) of the patient (P). The processor 52 includes a processing unit (e.g., a set of electronic circuits arranged to evaluate and execute software instructions using combinational logic circuitry or other similar circuitry) communicating with one or more memories. The processor 52 of the present example is mounted in a console 58, which includes operating controls 54 that include a keypad and/or a pointing device such as a mouse or trackball. A physician uses the operating controls 54 to interact with the processor 52 while performing the surgical procedure.

While not shown, the instrument may include a navigation sensor that is responsive to positioning within the alternating magnetic fields generated by the field generators 64. A coupling unit (not shown) may be secured to the proximal end of the instrument and may be configured to provide communication of data and other signals between the console 58 and the instrument. The coupling unit may provide wired or wireless communication of data and other signals.

In some versions, the navigation sensor of the instrument may comprise at least one coil at or near the distal end of the instrument. When such a coil is positioned within an alternating electromagnetic field generated by the field generators 64, the alternating magnetic field may induce electrical current in the coil, and this induced electrical current may be communicated as a position-indicative signal along the electrical conduit(s) in the instrument and further to the processor 52 via the coupling unit. This phenomenon may enable the IGS navigation system 50 to determine the location of the distal end of the instrument within a three-dimensional space (i.e., within the head (H) of the patient (P), etc.). To accomplish this, the processor 52 executes an algorithm to calculate location coordinates of the distal end of the instrument from the position related signals (e.g., from induced currents) of the coil(s) in the instrument. Thus, a navigation sensor may serve as a position sensor by generating signals indicating the real-time position of the sensor within three-dimensional space; or by otherwise indicating the real-time position of the sensor within three-dimensional space.

The processor 52 uses software stored in a memory of the processor 52 to calibrate and operate the IGS navigation system 50. Such operation includes driving the field generators 64, processing data from the instrument, processing data from the operating controls 54, and driving the display screen 56. In some implementations, operation may also include monitoring and enforcement of one or more safety features or functions of the IGS navigation system 50. The processor 52 is further operable to provide video in real time via the display screen 56, showing the position of the distal end of the instrument in relation to a video camera image of the patient's head (H), a CT scan image of the patient's head (H), and/or a computer-generated three-dimensional model of the anatomy within and adjacent to the patient's nasal cavity. The display screen 56 may display such images simultaneously and/or superimposed on each other during the surgical procedure. Such displayed images may also include graphical representations of instruments that are inserted in the patient's head (H), such that the operator may view the virtual rendering of the instrument at its actual location in real time. By way of example only, the display screen 56 may provide images in accordance with at least some of the teachings of U.S. Pat. No. 10,463,242, entitled “Guidewire Navigation for Sinuplasty,” issued Nov. 5, 2019, the disclosure of which is incorporated by reference herein, in its entirety. In the event that the operator is also using an endoscope, the endoscopic image may also be provided on the display screen 56.

The images provided through the display screen 56 may help guide the operator in maneuvering and otherwise manipulating instruments within the patient's head (H). It should also be understood that other components of a surgical instrument and other kinds of surgical instruments, as described below, may incorporate a navigation sensor like the navigation sensor described above.

II. Example of an ENT Instrument with a Distal Endoscope Cap

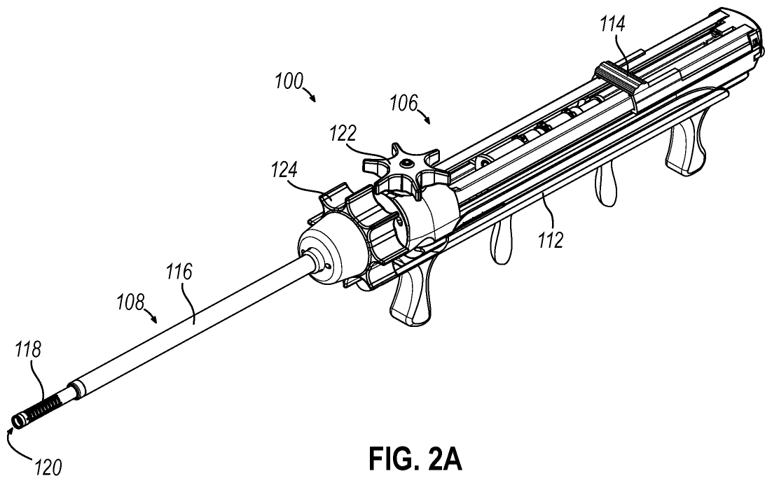

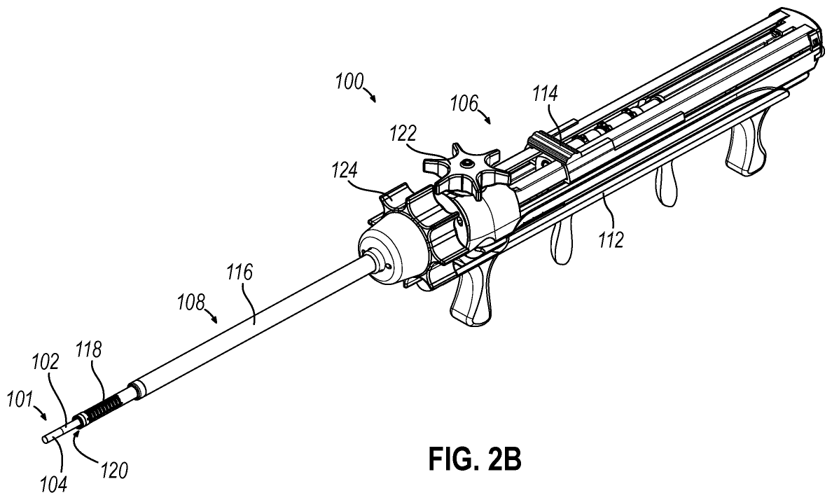

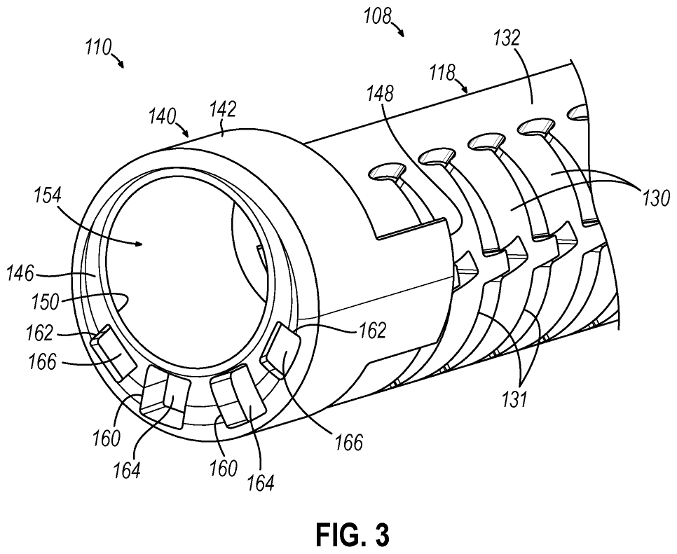

FIGS. 2A-3 show an example of an instrument 100 that may be used to guide a working element 101 (FIG. 2B) into an anatomical passageway, and to which an example of a distal endoscope cap 110 (FIG. 3) may be either removably or permanently attached. As shown in FIG. 2B, the working element 101 includes a shaft 102 and an end effector 104. In some versions, the working element 101 may include a dilation catheter. In this regard, the end effector 104 may have one or more balloons or other dilators, such that the instrument 100 may be used to guide the end effector 104 of the working element 101 into an anatomical passageway to thereby dilate the anatomical passageway. For instance, the instrument 100 and the working element 101 may be used for dilation of ostia of paranasal sinuses (e.g., to treat sinusitis), dilation of the larynx, dilation of the Eustachian tube, dilation of other passageways within the ear, nose, or throat, etc.

In addition, or alternatively, the working element 101 may include an electrical energy (e.g., RF energy and/or pulsed field DC energy, etc.) delivery catheter. In this regard, the end effector 104 may have one or more electrodes, such that the instrument 100 may be used to guide the end effector 104 of working element 101 into an anatomical passageway to deliver electrical energy to tissue in or near the anatomical passageway. For instance, the instrument 100 and the working element 101 may be used to ablate a nerve (e.g., a posterior nasal neve); ablate a turbinate; or ablate, electroporate (e.g., to promote absorption of therapeutic agents, etc.), or apply resistive heating to any other kind of anatomical structure in the head of a patient. It will be appreciated that the working element 101 may include any other suitable type of ENT treatment device.

The instrument 100 of this example includes a handle assembly 106 and a shaft assembly 108. The instrument 100 may be coupled with an inflation fluid source (not shown), which may be operable to selectively supply an inflation fluid to a balloon (not shown) of the end effector 104, for inflating the balloon to thereby dilate the anatomical passageway. In addition, or alternatively, the instrument 100 may be coupled with an ablation energy generator (not shown), which may be operable to generate ablation energy for delivery to tissue via electrodes (not shown) of the end effector 104 to thereby ablate, electroporate, or apply resistive heating to the tissue. Energy produced by the ablation energy generator may include, but is not limited to, radiofrequency (RF) energy or pulsed-field ablation (PFA) energy, including monopolar or bipolar high-voltage DC pulses as may be used to effect irreversible electroporation (IRE), or combinations thereof.

The handle assembly 106 of this example includes a body 112 and at least one slider 114. The body 112 is sized and configured to be grasped and operated by a single hand of an operator, such as via a power grip, a pencil grip, or any other suitable kind of grip. The slider 114 is operable to translate longitudinally relative to the body 112. The slider 114 is coupled with the working element 101 and is thus operable to translate the working device 101 longitudinally between a proximally retracted position (FIG. 2A) and a distally extended position (FIG. 2B). In some versions, another slider (not shown) may be operable to translate a guidewire (not shown) longitudinally for directing the working device 101 therealong.

The shaft assembly 108 of the present example includes a rigid portion 116, a flexible portion 118 distal to the rigid portion 116, and an open distal end 120. A pull-wire (not shown) is coupled with the flexible portion 118 and with a deflection control knob 122 of the handle assembly 106. The deflection control knob 122 is rotatable relative to the body 112, about an axis that is perpendicular to the longitudinal axis of the shaft assembly 108, to selectively retract the pull-wire proximally. As the pull-wire is retracted proximally, the flexible portion 118 bends and thereby deflects the distal end 120 laterally away from the longitudinal axis of the rigid portion 116. The deflection control knob 122, the pull-wire, and the flexible portion 118 thus cooperate to impart steerability to the shaft assembly 108. By way of example only, such steerability of the shaft assembly 108 may be provided in accordance with at least some of the teachings of U.S. Pat. Pub. No. 2021/0361912, entitled “Shaft Deflection Control Assembly for ENT Guide Instrument,” published Nov. 25, 2021, the disclosure of which is incorporated by reference herein, in its entirety. Other versions may provide some other kind of user input feature to drive steering of the flexible portion 118, instead of the deflection control knob 122. In some alternative versions, the deflection control knob 122 is omitted, and the flexible portion 118 is malleable. In still other versions, the entire length of the shaft assembly 108 is rigid.

The shaft assembly 108 is also rotatable relative to the handle assembly 106, about the longitudinal axis of the rigid portion 116. Such rotation may be driven via a rotation control knob 124, which is rotatably coupled with the body 112 of the handle assembly 106. Alternatively, the shaft assembly 108 may be rotated via some other form of user input; or may be non-rotatable relative to the handle assembly 106. It should also be understood that the example of the handle assembly 106 described herein is merely an illustrative example. The shaft assembly 108 may instead be coupled with any other suitable kind of handle assembly or other supporting body.

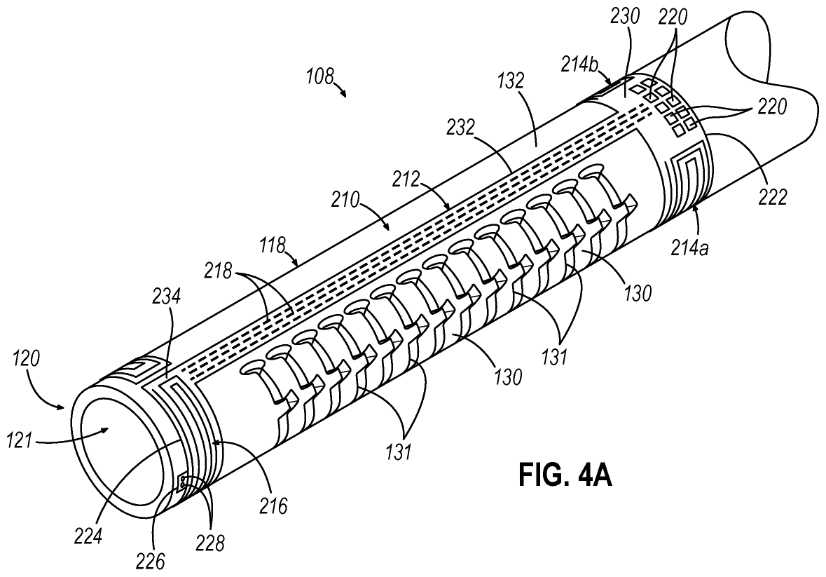

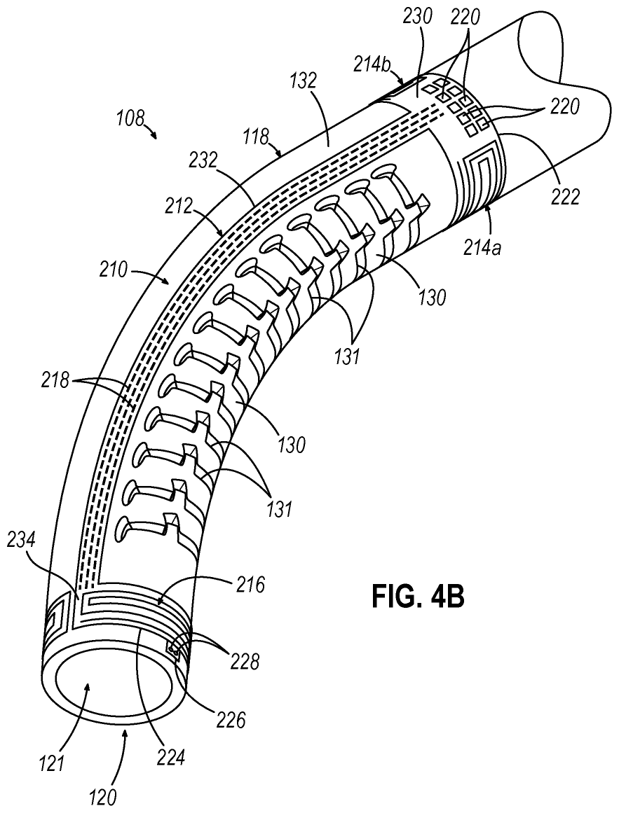

As shown in FIG. 3, the flexible portion (also referred to as a flexible guide shaft or a deflectable guide shaft) 118 of the shaft assembly 108 includes a linear array of articulating ribs 130 spaced apart from each other by generally C-shaped slots 131 and connected to each other by a resilient spine 132 for accommodating articulation of the articulating ribs 130 relative to the rigid portion 116 via the pull-wire described above. To that end, the flexible portion 118 may also include a pair of pull-wire coupling holes (not shown) or other features near an open distal end 120 for coupling with a distal end of the pull-wire. A working lumen 121 (FIGS. 4A-4B) extends longitudinally from an open proximal end (not shown) of the shaft assembly 108 all the way to the open distal end 120 and is configured to slidably receive the working element 101, such that the shaft assembly 108 may receive the working element 101 at the open proximal end, and such that the shaft assembly 108 may guide the working element 101 out through the open distal end 120. In some versions, the working lumen 121 may have a diameter of about 2.9 mm. the flexible portion 118 of the shaft assembly 108 may be formed of a metallic material, such as stainless steel and/or nitinol. In addition, or alternatively, the flexible portion 118 may be configured and operable in accordance with any one or more of the teachings of U.S. Pat. No. 11,376,401, entitled “Deflectable Guide for Medical Instrument,” issued Jul. 5, 2022, the disclosure of which is incorporated by reference herein, in its entirety.

With continuing reference to FIG. 3, the distal endoscope cap 110 is attachable to the open distal end 120 of shaft assembly 108 and is operable to provide visualization, navigation, and/or irrigation capabilities to the shaft assembly 108 while allowing the shaft assembly 108 to continue to be used to guide the working element 101 through the open distal end 120.

In this regard, the distal endoscope cap 110 of the present example includes a body 140 having a generally cylindrical hub 142 extending between a proximal surface (not shown) and a distal surface 146. In the example shown, the body 140 also has a pair of laterally-opposed coupling wings 148 extending proximally from the proximal surface of hub 142. Each of the wings 148 of the distal endoscope cap 110 includes a laterally inwardly-facing gripping surface (not shown) configured to frictionally engage a generally cylindrical outer surface of the flexible portion 118 of the shaft assembly 108 near the open distal end 120 for removably attaching the distal endoscope cap 110 to the open distal end 120. While gripping surfaces of the wings 148 have been described for frictionally engaging the cylindrical outer surface of the flexible portion 118, it will be appreciated that the distal endoscope cap 110 may be either removably or permanently attached to the open distal end 120 in any suitable manner, such as via adhesive, thermal bonding, welding, snap fit, or any other attachment techniques.

The distal endoscope cap 110 of the present example also includes a generally cylindrical bore 150 extending longitudinally between the proximal surface and the distal surface 146 of the hub 142 and configured to be axially aligned with the working lumen 121 of the shaft assembly 108 when the distal endoscope cap 110 is attached to the open distal end 120, such that the working element 101 may pass through the bore 150 as the working element 101 is guided through the distal open end 120. Thus, a working channel 154 may extend along the bore 150. In some versions, the bore 150 may have an inner cross-dimension (e.g., diameter) of between about 2.5 mm and about 2.9 mm. Alternatively, the bore 150 may have any other suitable inner cross-dimension.

As used herein, the term “axially aligned” should not be read as necessarily requiring that the central axis of the bore 150 must be coaxial with the central axis of the working lumen 121 of the shaft assembly 108. Instead, the term “axially aligned” should be read as including arrangements where the central axis of the working lumen 121 of the shaft assembly 108 passes through the bore 150, with the central axis of the working lumen 121 of the shaft assembly 108 being laterally offset from the central axis of the bore 150. “Axially aligned” thus includes any arrangements where a working element 101 that is advanced along the working lumen 121 of the shaft assembly 108 may ultimately pass through the bore 150. Of course, some versions of “axially aligned” arrangements may include arrangements where the central axis of the bore 150 is coaxial with the central axis of the working lumen 121 of the shaft assembly 108.

The distal endoscope cap 110 of the present example also includes an arch-shaped array of generally rectangular bores 160, 162 each extending longitudinally between the proximal surface and the distal surface 146 of the hub 142 and disposed about (e.g., below) the bore 150. More particularly, the distal endoscope cap 110 includes an inner pair of laterally-opposed bores 160 and an outer pair of laterally-opposed bores 162.

In the example shown, the distal endoscope cap 110 further includes a pair of imaging devices (also referred to as image sensors) in the form of cameras 164 received within the respective inner bores 160 and a pair of illuminating elements 166 received within the respective outer bores 162. The cameras 164 and the illuminating elements 166 are configured to cooperate with each other to provide visualization capabilities to the shaft assembly 108. By way of example only, the cameras 164 may be configured and operable in accordance with at least some of the teachings of U.S. Pat. No. 10,955,657, entitled “Endoscope with Dual Image Sensors,” issued Mar. 23, 2021, the disclosure of which is incorporated by reference herein; U.S. Pat. Pub. No. 2020/0196851, entitled “3D Scanning of Nasal Tract with Deflectable Endoscope,” published Jun. 25, 2020, the disclosure of which is incorporated by reference herein, in its entirety; and/or U.S. Pat. No. 11,457,981, entitled “Computerized Tomography (CT) Image Correction Using Position and Direction (P&D) Tracking Assisted Optical Visualization,” issued Oct. 4, 2022, the disclosure of which is incorporated by reference herein, in its entirety. In some cases, the centers of the cameras 164 may be spaced apart from each other by a distance of between about 1 mm and about 2 mm. Each of the cameras 164 may have a plurality of leads (not shown) on a proximal end thereof configured to be operatively coupled to the processor 52 and/or electrically coupled to a power source (not shown) via respective traces or wires extending proximally through the respective bore 160 and along the shaft assembly 108 to the coupling unit, for example.

The illuminating elements 166 are configured and operable to illuminate the field of view of the cameras 164. Each of the illuminating elements 166 is positioned outboard relative to the adjacent camera 164. In the present example, the illuminating elements 166 include LEDs. Each of the illuminating elements 166 may have a pair of leads (not shown) on a proximal end thereof configured to be electrically coupled to a power source (not shown) via respective traces or wires extending proximally through the respective bore 162 and along the shaft assembly 108 to the coupling unit, for example.

While the instrument 100 has been described for dilating an anatomical passageway and/or for delivering electrical energy to tissue within the ear, nose, or throat of a patient, it will be appreciated that the instrument 100 may be adapted to perform other surgical functions including, for example, diagnostic procedures, electrophysiology mapping, electrophysiology directed catheter guided surgery, and/or cardiac ablation procedures, such as via various other types of the working elements 101. By way of example only, the instrument 100 and/or the distal endoscope cap 110 may be configured and operable in accordance with at least some of the teachings of U.S. patent application Ser. No. 18/115,310, entitled “ENT Guide Shaft with Deflectable Tip and Distal Endoscope Cap,” filed Feb. 28, 2023, the disclosure of which is incorporated by reference herein, in its entirety.

III. Example of a Flex Circuit with Longitudinally-Spaced Position Sensors

In some instances, it may be desirable to provide the instrument 100 with a pair of proximal navigation sensors configured to generate position-related signals that are collectively indicative of the orientation of at least a portion of the shaft assembly 108 (e.g., a distal end of the rigid portion 116 and/or a proximal end of the flexible portion 118) about the longitudinal axis of the rigid portion 116 of the shaft assembly 108, also referred to as the “roll” of the portion of the shaft assembly 108. Such position-related signals may be provided by currents induced in one or more coils of the navigation sensors by an electromagnetic field generated by the field generators 64.

In addition, or alternatively, it may be desirable to provide the instrument 100 with at least one distal navigation sensor configured to generate position-related signals that are indicative of the position of the distal endoscope cap 110 and/or a distal portion of the shaft assembly 108 (e.g., a distal end of the flexible portion 118). Again, such position-related signals may be provided by currents induced in one or more coils of the navigation sensors by an electromagnetic field generated by the field generators 64.

In such an arrangement of navigation sensors, the sensors that are proximal to the flexible portion 118 may provide real-time “roll” orientation information of the region of the shaft assembly 108 proximal to flexible portion; the sensors that are proximal to the flexible portion 118 may also provide the real-time position information of the region of the shaft assembly 108 proximal to flexible portion in three-dimensional space; the sensor that is distal to the flexible portion 118 may also provide the real-time position information of the region of the shaft assembly 108 distal to flexible portion in three-dimensional space; and the combination of the sensors that are proximal to the flexible portion 118 and the sensor that is distal to the flexible portion 118 may together provide information indicating the real-time bend angle of the flexible portion 118.

As mentioned above, each of the cameras 164 and each of the illuminating elements 166 may have corresponding leads on a proximal end thereof configured to be operatively coupled to the processor 52 and/or electrically coupled to a power source (not shown) via respective traces or wires (not shown) extending proximally through the respective bore 160, 162 and along the shaft assembly 108 to the coupling unit, for example. In some instances, it may be desirable to route such traces or wires along the resilient spine 132 of the flexible portion 118 of the shaft assembly 108. For example, routing such traces or wires along the resilient spine 132 may allow such traces or wires to avoid interfering with the ability of the flexible portion 118 to bend. In addition, or alternatively, routing such traces or wires along the resilient spine 132 may allow such traces or wires to experience tension when the distal end 120 is deflected laterally away from the longitudinal axis of the rigid portion 116 (e.g., rather than experiencing compression when the distal end 120 is deflected laterally away from the longitudinal axis, which may otherwise be the case if such traces or wires were routed along the articulating ribs 130 and across the slots 131 opposite the resilient spine 132). It will be appreciated that tensioning of such traces or wires during deflection of the distal end 120 may assist with preventing such traces or wires from inadvertently contacting each other (e.g., shorting).

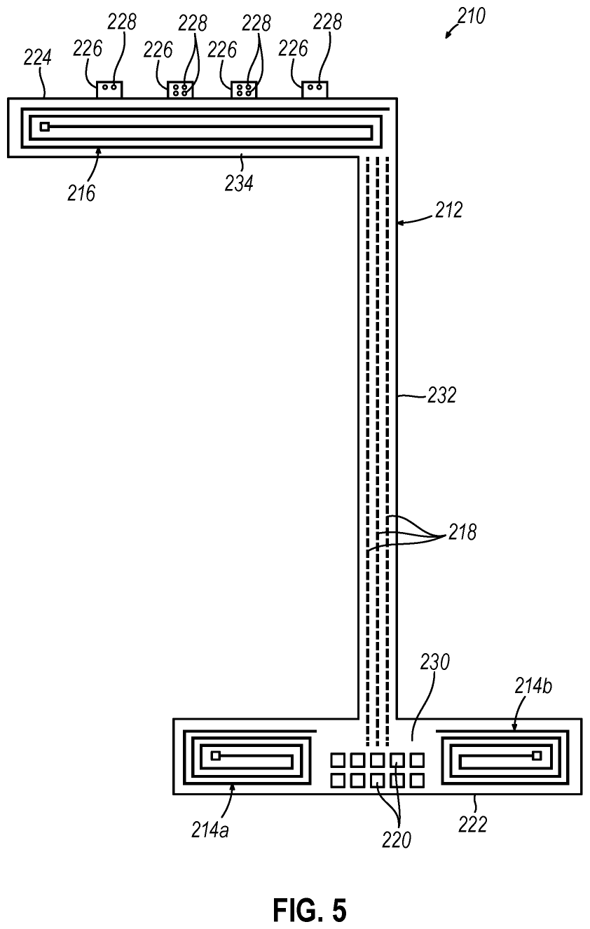

FIGS. 4A-5 show an example of a navigation sensor assembly 210 having such functionality. As shown in FIGS. 4A-4B, the navigation sensor assembly 210 is disposed on the shaft assembly 108 and is operable to provide navigation capabilities to the shaft assembly 108. More particularly, the navigation sensor assembly 210 is disposed along a generally cylindrical outer surface of the flexible portion 118 of the shaft assembly 108 in at least one generally curved configuration in which the navigation sensor assembly 210 is curved about the longitudinal axis of the flexible portion 118 of the shaft assembly 108, with a radius of curvature corresponding to that of the cylindrical outer surface of the flexible portion 118 to thereby conform to an outer circumference of the flexible portion 118. The transition from FIG. 4A to FIG. 4B shows the flexible portion 118 of the shaft assembly 108 bending from a straight configuration (FIG. 4A) to a bent configuration (FIG. 4B) and thereby deflecting the distal end 120 laterally away from the longitudinal axis of the rigid portion 116.

The navigation sensor assembly 210 of the present example is provided in the form of a flexible printed circuit board (PCB) and includes a generally L-shaped flex circuit substrate 212 with a plurality of navigation sensors 214a, 214b, 216 including a pair of laterally-adjacent proximal navigation sensors 214a, 214b and a distal navigation sensor 216, a plurality of traces 218, and a plurality of proximal leads (e.g., solder pads) 220 positioned thereon. As shown, the flexible substrate 212 extends longitudinally between proximal and distal ends 222, 224. In the example shown, a plurality of flexible tabs 226 having respective distal leads (e.g., solder pads) 228 positioned thereon extend distally from the distal end 224 of the substrate 212. In some versions, the tabs 226 and the substrate 212 may be integrally formed together as a unitary (e.g., monolithic) piece. The substrate 212 and/or the tabs 226 may be formed of an electrically-insulative, flexible plastic material such as polyimide or liquid crystal polymer (LCP), while the traces 218 and/or the leads 220, 228 may each be formed of an electrically-conductive, metallic material such as copper. In some versions, the substrate 212 is secured to an exterior surface of the flexible portion 118 of the shaft assembly 108 via an adhesive. Alternatively, the substrate 212 may be secured to the shaft assembly 108 in any other suitable fashion. The navigation sensor assembly 210 may have a relatively low profile, at least by comparison to traditional coil sensors. In some versions, the navigation sensor assembly 210 may have a thickness of approximately 50 microns.

The substrate 212 of the present example includes a proximal portion 230, an intermediate portion 232 extending distally from proximal portion 230, and a distal portion 234 extending laterally outwardly away from the intermediate portion 232, with the proximal navigation sensors 214a, 214b and the proximal leads 220 positioned on the proximal portion 230, the traces 218 positioned at least partially on each of the proximal, intermediate, and distal portions 230, 232, 234, and the distal navigation sensor 216 positioned on the distal portion 234. In the example shown, the proximal portion 230 of the substrate 212 extends laterally outwardly relative to each lateral side of the intermediate portion 232 such that the proximal portion 230 and the intermediate portion 232 collectively define a “T” shape, while the distal portion 234 of the substrate 212 extends laterally outwardly relative to only a single side of the intermediate portion 232 such that the distal portion 234 and the intermediate portion 232 collectively define an “L” shape, at least when the navigation sensor assembly 210 is in a flat configuration (FIG. 5).

As shown in FIGS. 4A-4B, the proximal portion 230 is sized and configured to wrap at least partially around the shaft assembly 108 at or near the proximal end of the flexible portion 118, such as proximally of the articulating ribs 130 and the slots 131; the distal portion 234 is sized and configured to wrap at least partially around the shaft assembly 108 at or near the distal end 120 of the shaft assembly 108, such as distally of the articulating ribs 130 and the slots 131; and the intermediate portion 232 is sized and configured to extend along the resilient spine 132 between the proximal and distal portions 230, 234. In some versions, at least the intermediate portion 232 includes a longitudinally extensible material.

In some versions, the flexible tabs 226 may be spaced apart from each other by distances corresponding to the distances between the cameras 164 and the illuminating elements 166, and/or may be arranged along the distal portion 234 so as to angularly align with the cameras 164 and the illuminating elements 166 relative to the longitudinal axis of the flexible portion 118 of the shaft assembly 108 (and/or relative to the longitudinal axis of the rigid portion 116 of the shaft assembly 108) when the distal portion 234 wraps at least partially around the shaft assembly 108 at or near the distal end 120. In other words, the flexible tabs 226 may be arranged so as to be angularly disposed about the longitudinal axis of the flexible portion 118 of the shaft assembly 108 relative to the longitudinal axis of the flexible portion 118 of the shaft assembly 108 (and/or relative to the longitudinal axis of the rigid portion 116 of the shaft assembly 108) at substantially same locations as those of the cameras 164 and the illuminating elements 166 when the distal portion 234 wraps at least partially around the shaft assembly 108 at or near the distal end 120.

In the example shown, the navigation sensors 214a, 214b, 216 are each defined by concentric loop portions of respective electrically-conductive traces formed (e.g., printed and/or embedded) on a top surface of the substrate 212, and are each operable to generate signals indicative of the position of the respective navigation sensor 214a, 214b, 216 and thereby indicative of the position of at least a portion (e.g., the flexible portion 118 of the shaft assembly 108) of the instrument 100 in three-dimensional space. In this regard, when the concentric loop portions of the respective electrically-conductive traces are positioned within an alternating electromagnetic field generated by the field generators 64, the alternating magnetic field may generate electrical current in the concentric loop portions and this electrical current may be communicated to the processor 52, such as via a coupling unit (not shown) electrically coupled to the navigation sensors 214a, 214b, 216. The position data generated by such position related signals may be processed by the processor 52 for providing a visual indication to the operator to show the operator where the shaft assembly 108 of the instrument 100 is located within the patient (P) in real time. Such a visual indication may be provided as an overlay on one or more preoperatively obtained images (e.g., CT scans) of the patient's anatomy.

In some versions, the traces that define each of the navigation sensors 214a, 214b, 216 may be concentric about a respective axis that is orthogonal to the axes of the other two navigation sensors 214a, 214b, 216, such that the navigation sensors 214a, 214b, 216 may collectively operate as a triple-axis sensor (TAS). For example, the distal portion 234 may wrap substantially entirely around the flexible portion 118 of the shaft assembly 108 at or near the distal end 120 such that the distal navigation sensor 216 may likewise wrap substantially entirely around the flexible portion 118, so that the traces that define the distal navigation sensor 216 may be concentric about the longitudinal axis of the flexible portion 118; while the proximal portion 230 may wrap partially around the shaft assembly 108 at or near the proximal end of the flexible portion 118 such that the proximal navigation sensors 214a, 214b may each only wrap partially around the shaft assembly 108 and may be spaced apart from each other so that the traces that define each of the proximal navigation sensors 214a, 214b may be concentric about a respective transverse axis, with the transverse axes being orthogonal to the longitudinal axis of the flexible portion 118 and orthogonal to each other.

As shown, the proximal navigation sensors 214a, 214b are arranged on the proximal portion 230 such that first proximal navigation sensor 214a is positioned laterally outwardly of a first side of intermediate portion 232 and second proximal navigation sensor 214b is positioned laterally outwardly of a second side of intermediate portion 232 opposite the first side, with the proximal leads 220 being arranged generally in-line with the intermediate portion 232. The proximal navigation sensors 214a, 214b may each be directly electrically coupled to the respective proximal leads 220. In the example shown, the traces 218 extend distally from the respective proximal leads 220 on the proximal portion 230 and extend longitudinally along the intermediate portion 232, and may further extend laterally at least partially along the distal portion 234 to respective ones of the distal leads 228 or the distal navigation sensor 216, such that the distal navigation sensor 216 may be electrically coupled to the respective proximal leads 220 via the respective traces 218, and/or such that the distal leads 228 may each be electrically and/or operatively coupled to the respective proximal leads 220 via the respective traces 218.

In this regard, the distal leads 228 of each of the tabs 226 are configured to electrically couple and/or operatively couple to a corresponding one of the cameras 164 or the illuminating elements 166, such that the cameras 164 may each be electrically and/or operatively coupled to the respective proximal leads 220 via the respective traces 218 and the distal leads 228, and such that the illuminating elements 166 may each be electrically coupled to the respective proximal leads 220 via the respective traces 218 and the distal leads 228. For example, each of the tabs 226 may be angularly disposed about a longitudinal axis of the shaft assembly 108 at a substantially same location as that of the corresponding one of the cameras 164 or illuminating elements 166, such that the distal leads 228 of each of the tabs 226 may be directly electrically coupled and/or operatively coupled to the respective camera leads or illuminating element leads of the corresponding one of the cameras 164 or illuminating elements 166 (e.g., without intervening electrical wires). More particularly, each outer tab 226 carrying two distal leads 228 may be angularly aligned with a respective illuminating element 166 relative to the longitudinal axis of the shaft assembly 108 to place the respective distal leads 228 in direct electrical communication with the respective illuminating element leads of the respective illuminating element 166, while each inner tab 226 carrying four distal leads 228 may be angularly aligned with a respective camera 164 relative to the longitudinal axis of the shaft assembly 108 to place the respective distal leads 228 in direct electrical communication with respective camera leads of the respective camera 164.

The proximal leads 220 may, in turn, each be operatively coupled to the processor 52 and/or electrically coupled to a power source (not shown) via respective wires (not shown) extending proximally from the navigation sensor assembly 210 along the shaft assembly 108 to the coupling unit, for example, so that position related signals may be transmitted from the navigation sensors 214a, 214b, 216 to the coupling unit, power may be supplied to the cameras 164 and the illuminating elements 166, and image signals may be transmitted from the cameras 164 to the coupling unit via such wires. In some cases, such wires may be bundled together in one or more cables. In addition, or alternatively, such wires may be routed proximally from the navigation sensor assembly 210 through the rigid portion 116 of shaft assembly 108 so that such wires may be substantially concealed within the rigid portion 116. It will be appreciated that the particular numbers of the proximal leads 220, the traces 218, and the distal leads 228 shown are for illustrative purposes only, and that any suitable numbers of the proximal leads 220, the traces 218, the distal leads 228, and corresponding wires may be used.

As shown in FIG. 5, the navigation sensor assembly 210 may initially have a generally flat configuration, such as when the substrate 212 is initially formed and/or during the initial positioning of the navigation sensors 214a, 214b, 216 thereon. As shown in FIG. 4A, the navigation sensor assembly 210 may assume a laterally curved, longitudinally straight configuration in which the proximal and distal portions 230, 234 are curved downwardly from the intermediate portion 232. In this manner, the navigation sensor assembly 210 may be curved about the longitudinal axis of the flexible portion 118 of the shaft assembly 108 with a radius of curvature corresponding to that of the cylindrical outer surface of the flexible portion 118 when disposed on the flexible portion 118 to thereby conform to the outer circumference of flexible portion 118. As shown in FIG. 4B, the navigation sensor assembly 210 may assume a laterally curved, longitudinally bent configuration in which the proximal and distal portions 230, 234 are curved downwardly from the intermediate portion 232 with the navigation sensor assembly 210 at least partially deflecting from the longitudinal direction. The navigation sensor assembly 210 may be in its laterally curved, longitudinally straight configuration when the flexible portion 118 of the shaft assembly 108 is in its straight configuration, and the navigation sensor assembly 210 may be in its laterally curved, longitudinally bent configuration when the flexible portion 118 of the shaft assembly 108 is in its bent configuration. In this manner, navigation sensor assembly 210 may accommodate the bending of the flexible portion 118 between its straight and bent configurations, such that navigation of the flexible portion 118 may be performed irrespective of whether the flexible portion 118 is in its straight or bent configurations.

In the example shown, the distal navigation sensor 216 is positioned at or near the distal end 120 of the shaft assembly 108 for facilitating navigation of the distal end 120, while the proximal navigation sensors 214a, 214b are positioned at or near the proximal end of the flexible portion 118 for facilitating determining the orientation of the proximal end of the flexible portion 118 about the longitudinal axis of the rigid portion 116. More particularly, the distal navigation sensor 216 is positioned distal of the articulating ribs 130 and the slots 131 such that the distal navigation sensor 216 deflects laterally away from the longitudinal axis of the rigid portion 116 as the distal end 120 is deflected laterally away from the longitudinal axis of the rigid portion 116. Conversely, the proximal navigation sensors 214a, 214b are positioned proximal of the articulating ribs 130 and the slots 131 such that the proximal navigation sensors 214a, 214b do not deflect laterally away from the longitudinal axis of the rigid portion 116 as the distal end 120 is deflected laterally away from the longitudinal axis of the rigid portion 116. Thus, the position data from the distal navigation sensor 216 may be used to determine the position of the distal end of the flexible portion 118, while the position data from the proximal navigation sensors 214a, 214b may be used to determine the orientation or “roll” of the proximal end of the flexible portion 118 about the longitudinal axis of the rigid portion 116 of the shaft assembly 108 (in addition to providing signals indicating the position of the proximal end of the flexible portion 118 in three-dimensional space). In some instances, the position data from the proximal navigation sensors 214a, 214b may be compared with the position data from the distal navigation sensor 216 to precisely determine the degree of lateral deflection of the distal end 120 in relation to the frame of reference of the IGS navigation system 50. Of course, the navigation sensors 214a, 214b, 216 may be positioned at any other suitable locations relative to components of the instrument 100 for which navigation is desired; and may be used in any other suitable ways.

As shown in FIGS. 4A-4B, the intermediate portion 232 of the substrate 212 extends along the resilient spine 132 of the flexible portion 118 of the shaft assembly 108 such that the traces 218 likewise extend along the resilient spine 132. In this manner, the traces 218 may avoid interfering with the ability of the flexible portion 118 to bend. In addition, or alternatively, the traces 218 may experience tension when the distal end 120 is deflected laterally away from the longitudinal axis of the rigid portion 116 to assist with preventing the traces 218 from inadvertently contacting each other (e.g., shorting) during deflection of the distal end 120. As noted above, at least the intermediate portion 232 of the substrate 212 may include an extensible material to further accommodate any stretching of the intermediate portion 232 that might occur during bending of the flexible portion 118. In addition, the traces 218 may be configured to accommodate any stretching that might occur along the intermediate portion 232 during bending of the flexible portion 118. For instance, the traces 218 may be formed in wavy or serpentine configurations, allowing the effective lengths of the traces 218 to increase during bending of the flexible portion 118.

While the navigation sensor assembly 210 of the present example is disposed along a generally cylindrical outer surface of the flexible portion 118 of the shaft assembly 108, the navigation sensor assembly 210 may alternatively be disposed along a generally cylindrical inner surface of the flexible portion 118 of shaft assembly 108 in at least one generally curved configuration in which the navigation sensor assembly 210 is curved about the longitudinal axis of the flexible portion 118 of the shaft assembly 108 with a radius of curvature corresponding to that of the cylindrical inner surface of the flexible portion 118 to thereby conform to an inner circumference of the flexible portion 118. In addition to the foregoing, the navigation sensor assembly 210 may be configured and operable in accordance with at least some of the teachings of U.S. Pub. No. 2022/0257093, entitled “Flexible Sensor Assembly for ENT Instrument,” published Aug. 18, 2022, the disclosure of which is incorporated by reference herein, in its entirety.

### IV. Examples of Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### EXAMPLE 1

An apparatus, comprising: (a) a shaft assembly including a proximal portion and a distal portion, the distal portion being configured to enable lateral deflection of the distal end away from or toward a longitudinal axis defined by the proximal portion; (b) a distal navigation sensor positioned at or near the distal end of the distal portion, the distal navigation sensor being configured to indicate a position of the distal end of the distal portion in three-dimensional space; and (c) a pair of proximal navigation sensors positioned at or near a proximal end of the distal portion, the pair of proximal navigation sensors being configured to indicate a roll orientation of the proximal end of the distal portion about the longitudinal axis.

### EXAMPLE 2

The apparatus of Example 1, the distal portion including a linear array of articulating ribs.

### EXAMPLE 3

The apparatus of Example 2, the distal navigation sensor being positioned distal of the linear array of articulating ribs.

### EXAMPLE 4

The apparatus of any of Examples 2 through 3, the pair of proximal navigation sensors being positioned proximal of the linear array of articulating ribs.

### EXAMPLE 5

The apparatus of any of Examples 2 through 4, the articulating ribs being connected to each other by a resilient spine.

### EXAMPLE 6

The apparatus of Example 5, further comprising at least one electrically conductive element extending along the resilient spine.

### EXAMPLE 7

The apparatus of Example 6, the at least one electrically conductive element being electrically coupled to the distal navigation sensor.

### EXAMPLE 8

The apparatus of any of Examples 1 through 7, further comprising a flexible substrate secured to the distal portion, each of the proximal and distal navigation sensors being disposed on the flexible substrate to define a navigation sensor assembly.

### EXAMPLE 9

The apparatus of Example 8, the navigation sensor assembly including a plurality of proximal leads, each of the proximal and distal navigation sensors being electrically coupled to respective proximal leads of the plurality of proximal leads.

### EXAMPLE 10

The apparatus of any of Examples 8 through 9, the navigation sensor assembly including at least one distal lead configured to be electrically coupled to at least one of an image sensor or an illuminating element.

### EXAMPLE 11

The apparatus of Example 10, the at least one distal lead being positioned distal of the distal navigation sensor.

### EXAMPLE 12

The apparatus of any of Examples 10 through 11, further comprising at least one of an image sensor or an illuminating element, the at least one distal lead being electrically coupled to the at least one of an image sensor or an illuminating element.

### EXAMPLE 13

The apparatus of Example 12, the at least one distal lead being angularly aligned with the at least one of an image sensor or an illuminating element relative to the longitudinal axis.

### EXAMPLE 14

The apparatus of any of Examples 12 through 13, the at least one of an image sensor or an illuminating element including an image sensor and an illuminating element.

### EXAMPLE 15

The apparatus of Example 14, the at least one distal lead including at least one first distal lead electrically coupled to the image sensor and at least one second distal lead electrically coupled to the illuminating element.

### EXAMPLE 16

The apparatus of any of Examples 1 through 15, the proximal portion of the shaft assembly being rigid.

### EXAMPLE 17

The apparatus of any of Examples 1 through 16, the distal portion of the shaft assembly being flexible.

### EXAMPLE 18

The apparatus of any of Examples 1 through 17, the distal portion having a distal end sized and configured to fit in an anatomical passageway in an ear, nose, or throat of a patient.

### EXAMPLE 19

The apparatus of any of Examples 1 through 18, the distal navigation sensor being configured indicate a position of the distal end of the distal portion in three-dimensional space by generating signals indicative of a position of the distal end of the distal portion in three-dimensional space.

### EXAMPLE 20

The apparatus of any of Examples 1 through 19, the pair of proximal navigation sensors being configured indicate a roll orientation of the proximal end of the distal portion about the longitudinal axis by generating signals indicative of a roll orientation of the proximal end of the distal portion about the longitudinal axis.

### EXAMPLE 21

An apparatus, comprising: (a) a shaft assembly including a proximal portion and a flexible distal portion, the distal portion being configured to enable lateral deflection of the distal end away from or toward a longitudinal axis defined by the proximal portion; and (b) a navigation sensor assembly extending along the distal portion, the navigation sensor assembly including: (i) a flexible substrate having a proximal portion, a distal portion, and an intermediate portion extending between the proximal and distal portions, (ii) a pair of laterally-adjacent proximal navigation sensors disposed on the proximal portion of the flexible substrate, (iii) a distal navigation sensor disposed on the distal portion of the flexible substrate, (iv) at least one distal lead configured to be electrically coupled to at least one of an image sensor or an illuminating element, and (v) at least one electrically conductive element extending along the intermediate portion, the at least one electrically conductive element being electrically coupled to at least one of the distal navigation sensor or the at least one distal lead.

### EXAMPLE 22

The apparatus of Example 21, the distal portion including a linear array of articulating ribs.

### EXAMPLE 23

The apparatus of Example 22, the distal navigation sensor being positioned distal of the linear array of articulating ribs, the pair of proximal navigation sensors being positioned proximal of the linear array of articulating ribs.

### EXAMPLE 24

The apparatus of any of Examples 22 through 23, the articulating ribs being connected to each other by a resilient spine, the intermediate portion of the flexible substrate extending along the resilient spine.

### EXAMPLE 25

The apparatus of any of Examples 21 through 24, the proximal portion of the shaft assembly being rigid.

### EXAMPLE 26

The apparatus of any of Examples 21 through 25, the distal portion of the shaft assembly being flexible.

### EXAMPLE 27

The apparatus of any of Examples 21 through 26, the distal portion having a distal end sized and configured to fit in an anatomical passageway in an ear, nose, or throat of a patient.

### EXAMPLE 28

The apparatus of any of Examples 21 through 27, the distal navigation sensor being configured indicate a position of the distal end of the distal portion in three-dimensional space.

### EXAMPLE 29

The apparatus of Example 28, the distal navigation sensor being configured indicate a position of the distal end of the distal portion in three-dimensional space by generating signals indicative of a position of the distal end of the distal portion in three-dimensional space.

### EXAMPLE 30

The apparatus of any of Examples 21 through 29, the pair of proximal navigation sensors being configured indicate a roll orientation of the proximal end of the distal portion about the longitudinal axis.

### EXAMPLE 31

The apparatus of any of Examples 21 through 30, the pair of proximal navigation sensors being configured indicate a roll orientation of the proximal end of the distal portion about the longitudinal axis by generating signals indicative of a roll orientation of the proximal end of the distal portion about the longitudinal axis.

### EXAMPLE 32

An apparatus, comprising: (a) a shaft assembly including a proximal portion and a distal portion, the distal portion being configured to enable lateral deflection of the distal end away from or toward a longitudinal axis defined by the proximal portion; (b) an image sensor secured to the distal end of the shaft assembly for visualizing an anatomical structure; (c) an illuminating element secured to the distal end of the shaft assembly for illuminating a field of view of the image sensor; and (d) a navigation sensor assembly extending along the distal portion, the navigation sensor assembly including: (i) at least one navigation sensor, and (ii) at least one distal lead configured to be electrically coupled to at least one of the image sensor or the illuminating element, the at least one distal lead being angularly aligned with the at least one of the image sensor or the illuminating element relative to the longitudinal axis.

### EXAMPLE 33

The apparatus of Example 32, the proximal portion of the shaft assembly being rigid.

### EXAMPLE 34

The apparatus of any of Examples 32 through 33, the distal portion of the shaft assembly being flexible.

### EXAMPLE 35

The apparatus of any of Examples 32 through 34, the distal portion having a distal end sized and configured to fit in an anatomical passageway in an ear, nose, or throat of a patient.

### EXAMPLE 36

The apparatus of any of Examples 32 through 35, the at least one navigation sensor comprising a distal navigation sensor and at least two proximal navigation sensors.

### EXAMPLE 37

The apparatus of Example 36, the distal navigation sensor being configured indicate a position of the distal end of the distal portion in three-dimensional space.

### EXAMPLE 38

The apparatus of Example 37, the distal navigation sensor being configured indicate a position of the distal end of the distal portion in three-dimensional space by generating signals indicative of a position of the distal end of the distal portion in three-dimensional space.

### EXAMPLE 39

The apparatus of any of Examples 36 through 38, the at least two proximal navigation sensors being configured indicate a roll orientation of the proximal end of the distal portion about the longitudinal axis.

### EXAMPLE 40

The apparatus of any of Example 39, the at least two proximal navigation sensors being configured indicate a roll orientation of the proximal end of the distal portion about the longitudinal axis by generating signals indicative of a roll orientation of the proximal end of the distal portion about the longitudinal axis.

### V. Miscellaneous

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those skilled in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility or by a user immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

1. An apparatus, comprising:
a shaft assembly including a proximal portion and a distal portion, the distal portion being configured to enable lateral deflection of the distal end away from or toward a longitudinal axis defined by the proximal portion;
a distal navigation sensor positioned at or near the distal end of the distal portion, the distal navigation sensor being configured to indicate a position of the distal end of the distal portion in three-dimensional space; and
a pair of proximal navigation sensors positioned at or near a proximal end of the distal portion, the pair of proximal navigation sensors being configured to indicate a roll orientation of the proximal end of the distal portion about the longitudinal axis.

2. The apparatus of claim 1, wherein the distal portion includes a linear array of articulating ribs.

3. The apparatus of claim 2, wherein the distal navigation sensor is positioned distal of the linear array of articulating ribs.

4. The apparatus of claim 2, wherein the pair of proximal navigation sensors is positioned proximal of the linear array of articulating ribs.

5. The apparatus of claim 2, wherein the articulating ribs is connected to each other by a resilient spine.

6. The apparatus of claim 5, further comprising at least one electrically conductive element extending along the resilient spine.

7. The apparatus of claim 6, wherein the at least one electrically conductive element is electrically coupled to the distal navigation sensor.

8. The apparatus of claim 1, further comprising a flexible substrate secured to the distal portion, wherein each of the proximal and distal navigation sensors is disposed on the flexible substrate to define a navigation sensor assembly.

9. The apparatus of claim 8, wherein:
the navigation sensor assembly includes a plurality of proximal leads, and
each of the proximal and distal navigation sensors is electrically coupled to respective proximal leads of the plurality of proximal leads.

10. The apparatus of claim 8, wherein the navigation sensor assembly includes at least one distal lead configured to be electrically coupled to at least one of an image sensor or an illuminating element.

11. The apparatus of claim 10, wherein the at least one distal lead is positioned distal of the distal navigation sensor.

12. The apparatus of claim 10, further comprising at least one of an image sensor or an illuminating element, wherein the at least one distal lead is electrically coupled to the at least one of an image sensor or an illuminating element.

13. The apparatus of claim 12, wherein the at least one distal lead is angularly aligned with the at least one of an image sensor or an illuminating element relative to the longitudinal axis.

14. The apparatus of claim 12, wherein the at least one of an image sensor or an illuminating element includes an image sensor and an illuminating element.

15. The apparatus of claim 14, wherein the at least one distal lead includes at least one first distal lead electrically coupled to the image sensor and at least one second distal lead electrically coupled to the illuminating element.

16. The apparatus of claim 1, wherein the proximal portion of the shaft assembly is rigid.

17. The apparatus of claim 1, wherein the distal portion of the shaft assembly is flexible.

18. The apparatus of claim 1, wherein the distal portion has a distal end sized and configured to fit in an anatomical passageway in an ear, nose, or throat of a patient.

19. The apparatus of claim 1, wherein the distal navigation sensor is configured to indicate a position of the distal end of the distal portion in three-dimensional space by generating signals indicative of a position of the distal end of the distal portion in three-dimensional space.

20. The apparatus of claim 1, wherein the pair of proximal navigation sensors is configured to indicate a roll orientation of the proximal end of the distal portion about the longitudinal axis by generating signals indicative of a roll orientation of the proximal end of the distal portion about the longitudinal axis.
